(19) United States
(12) Patent Application Publication
(10) Pub. No.: US 2008/0041374 A1
(43) Pub. Date: Feb. 21, 2008

(54) Hands-free chin lift and airway support device

(76) Inventor: Joan M. Reddick, Palm Harbor, FL (US)

Correspondence Address: BINGHAM MCCUTCHEN LLP, 2020 K Street, N.W., Intellectual Property Department, WASHINGTON, DC, 20006, US

(21) Appl. No.: 11/826,887

(22) Filed: Jul. 19, 2007

Related U.S. Application Data
(63) Continuation-in-part of application No. 11/142,228, filed on Jun. 2, 2005, now abandoned.
Application No. 11/142,228 is a continuation of application No. 10/366,761, filed on Feb. 14, 2003, now Pat. No. 6,969,366.

(51) Int. Cl. A61M 16/00

(52) U.S. Cl. 128/200.26; 128/848

(57) ABSTRACT

A support device having a chin rest with an upper convex portion, a malleable shaft, and a base configured with a lower convex portion are provided. The device is configured to maintain patency to the airway of a person with a decreased level of consciousness. The head of the person is positioned such that airway patency is confirmed. The device is then positioned such that airway patency of the person is maintained as needed.

### CROSS-REFERENCE TO RELATED APPLICATIONS

This application is a continuation-in-part of application having Ser. No. 11/142,228, filed Jun. 2, 2005, which is a continuation of application having Ser. No. 10/366,761, filed Feb. 14, 2003, now U.S. Pat. No. 6,969,366.

### FIELD OF INVENTION

This invention relates to anesthesia delivery, specifically to an apparatus and method for supporting the mandible to prevent airway obstruction.

### BACKGROUND OF THE INVENTION

Total Intravenous Anesthesia (TIVA) is the commonly used anesthetic technique consisting of delivering drugs into the bloodstream in combination with a local anesthetic infiltration by the surgeon at the operative site. TIVA anesthesia is often used in combination with regional anesthesia, such as spinals, epidurals, and peripheral nerve blocks, which also provide temporary loss of feeling and movement at the operative site. The risk of using TIVA anesthesia is that upper airway obstruction may occur due to respiratory depression.

During surgery, respiratory depression can occur in any person whose level of consciousness is decreased due to sedation from TIVA anesthesia. Respiratory depression in the unconscious person is the result of loss of tonicity of the submandibular muscles, which provide direct support of the tongue and indirect support to the epiglottis. As a result of this loss of tonicity, posterior displacement of the tongue may occlude the airway at the level of the pharynx, and the epiglottis may occlude the airway at the level of the larynx. Thus, to prevent airway obstruction, the anesthetist must achieve proper airway positioning in the patient to maintain airway patency. The basic technique for maintaining patency or opening the airway is the head-tilt with anterior displacement of the mandible (chin-lift or jaw-thrust maneuver).

To perform this technique the anesthetist will first attempt the chin lift maneuver, which consists of manually lifting the chin upwards. This maneuver provides maintenance of proper head tilt and anterior displacement of the mandible resulting in proper alignment of the airway structures, which contributes to patient air exchange. Another option is the jaw-thrust maneuver, which is performed by placing one's hands at both sides of the mandible laterally and thrusting the jaw forward. Both methods require the anesthetist to support the patient's head manually throughout the duration of the surgery.

In some TIVA anesthesia cases, an oropharyngeal or nasopharyngeal airway may be necessary to maintain airway patency. An oropharyngeal airway is a plastic, disposable, semi-circular shaped device that, when in proper position, will hold the tongue away from the posterior wall of the pharynx. However, even with the use of this device, proper head position must be maintained using either the chin-lift or jaw-thrust maneuver to keep the airway patent. The nasopharyngeal airway is an uncuffed tube made of soft rubber or plastic. Its use is indicated when the insertion of the oral airway is technically difficult or if the oral airway provides only partial relief of the airway obstruction. The airway is lubricated with a water-soluble lubricant and gently inserted close to the midline along the floor of the nostril into the posterior pharynx behind the tongue. Again, it is important to maintain head-tilt with anterior displacement of the mandible by chin-lift and, if necessary, jaw thrust when using the oropharyngeal or nasopharyngeal airway.

Surgical procedures using TIVA anesthesia can range from fifteen minutes to as long as two hours. The anesthetist must continuously have to administer sedative medications, and assess patient response to those medications as well as monitor and document vital signs on the patient's chart. Accordingly, if the anesthetist must physically perform the chin lift maneuver throughout the duration of the surgical procedure in order to maintain patency of the airway, the additional responsibilities of monitoring, documentation, and medication administration become more cumbersome. Additionally, factors, such as the position of the patient, often make the performance of the chin-lift maneuver awkward. The anesthetist may also become unnecessarily fatigued and/or stiff as a result of laboriously maintaining constant pressure upon the patient's chin.

In some cases, when it is obvious that maintaining continuous pressure on the mandible will be too taxing upon the anesthesia provider, he or she will choose to use general anesthesia instead of TIVA anesthesia to anesthetize the patient. General anesthesia carries the risk of major complications including death, myocardial infarction, and stroke, and it also is associated with less serious complications such as vomiting, sore throat, headache, shivering, and delayed return to normal mental functioning.

Accordingly, there is a need for a device that enables the anesthetist to more efficiently provide patient care during TIVA anesthesia. There is a need for this device to make it unnecessary for the anesthetist to continuously perform the chin-lift maneuver manually. There is a need for the device to remove the impediment of the anesthetist's being forced to maintain continuous physical contact with the patient's mandible at all times and allow the anesthetist unrestricted movement around the patient's bed. There is a need for the device to facilitate the anesthetist in delivering medications and charting vital signs during surgery. There is a need for the device to render unimportant operating room table placement. There is a need for the device to prevent the anesthetist from becoming unnecessarily fatigued and/or stiff as a result of laboriously maintaining constant pressure upon the patient's chin. There is a need for the device to prevent the anesthesia provider from being forced to induce general anesthesia in the patients to alleviate the difficulties to him/herself, which results in decreased risk of complications such as sore throat, increased nausea, and injury to teeth. There is a need for portions of the device to be clean and disposable. There is a need for a method that provides hands-free support of the mandible allowing proper alignment of anatomical structures of the airway, resulting in optimal air exchange, thereby preventing and/or resolving obstruction. There is a need for a method of assembling a device that provides hands-free support of the mandible allowing the proper alignment of the anatomical structures of the airway.

### SUMMARY OF THE INVENTION

The present invention is a hands free chin lift and airway support device. It is designed to provide support for the head of a patient when the muscles supporting the head are in a state of relaxation due to anesthesia delivered during a medical procedure. In an embodiment of the present invention, the device will have a chin rest or chin support portion that has an upper convex portion configured to be positioned and engage the patient under the chin or sub-mental so the patient's head position remains constant during movements that may occur during a medical procedure. The chin support portion also includes a lower portion configured to removably couple to a first end of a shaft support portion. The shaft support portion of the device will consist of a material that will be flexible and malleable and will retain shape when adjusted. The shaft support portion of the device will transfer the weight of a patient's head to a base portion. The base portion will to the patient's chest both by adhesive and/or by the friction caused by the transfer of force from the patient's head to the chin support portion.

In an embodiment of the present invention, the base portion includes a lower convex portion that is positioned on and engages the patient's chest.

In an embodiment of the present invention, the base support portion includes an upper portion configured to removably couple to a second end of the shaft support portion.

In an embodiment of the present invention, the lower convex portion of the base portion is configured with a thin film of adhesive having adherence to the skin at least sufficient to prevent the base portion from sliding on the patient's manubrium.

In an embodiment of the present invention, the upper convex portion of the chin support portion is configured with a thin film of adhesive having adherence to the skin at least sufficient to prevent the chin support portion from sliding on the patient's chin.

### DETAILED DESCRIPTION OF THE DRAWINGS

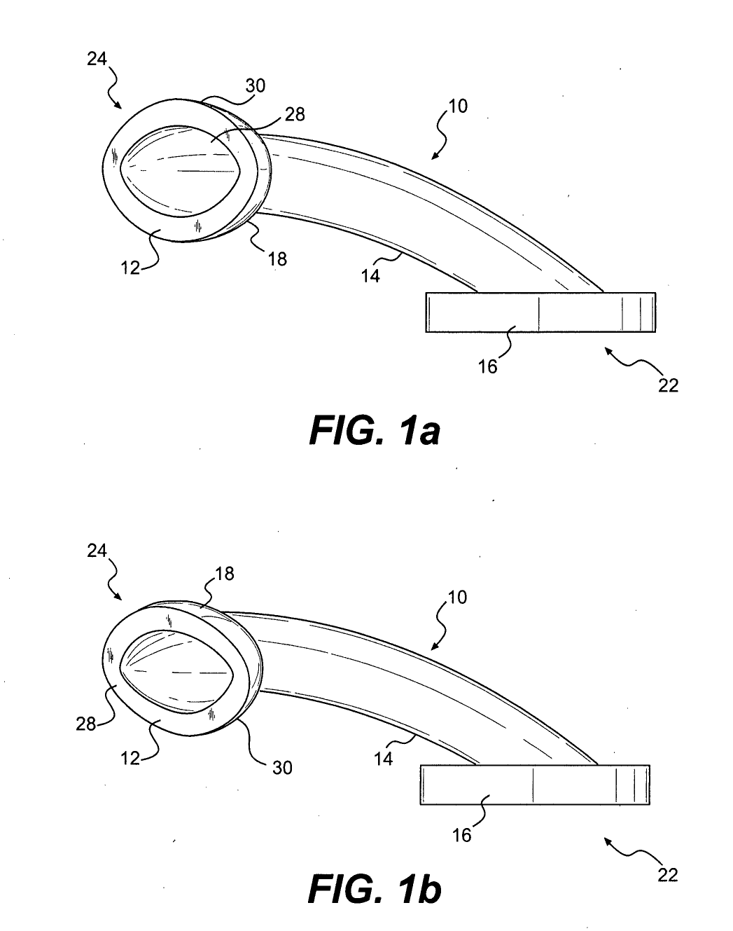

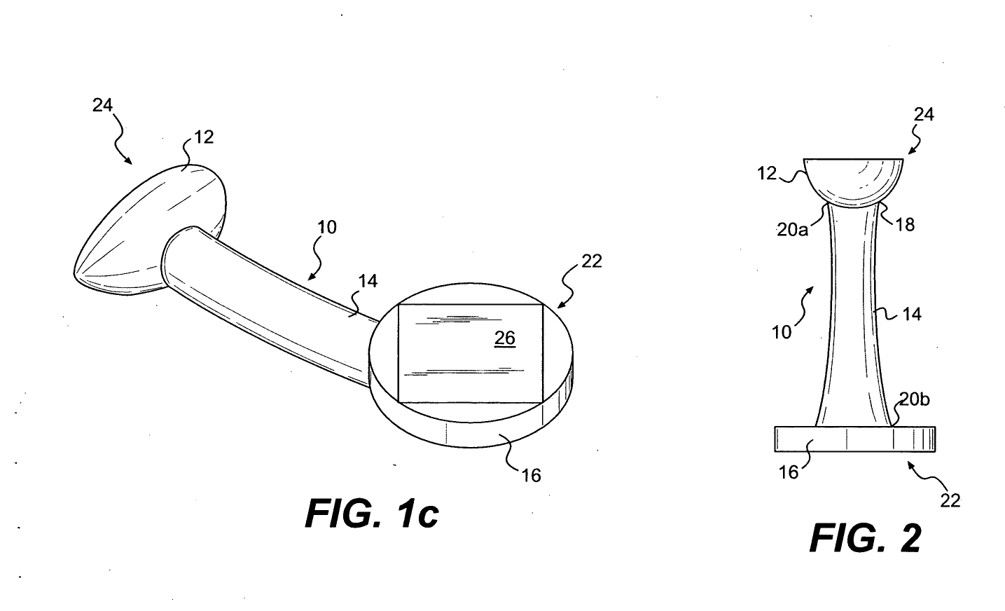

FIG. 1a is a perspective view of the hands free chin lift and airway support device shown with the shaft shown bent at the anticipated use angle;

FIG. 1b is a perspective view of the hands free chin lift and airway support device with the chin rest bend downward;

FIG. 1c is a perspective view of the bottom of the hands free chin lift and airway support device;

FIG. 2 shows the device in an upright position at rest, such that no internal stress is present in the device;

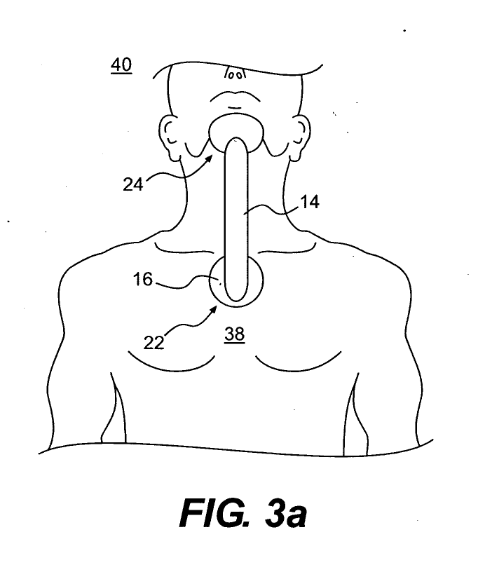

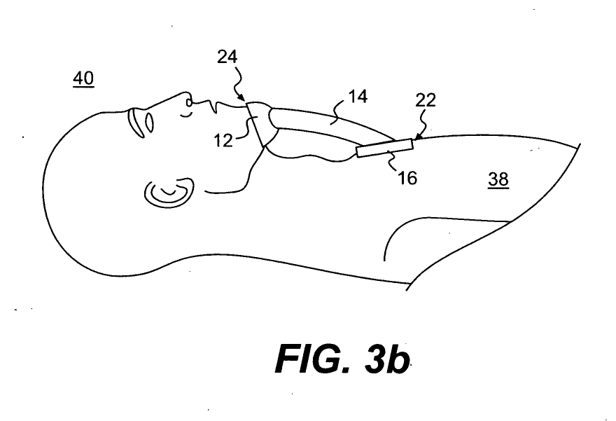

FIG. 3a shows a top view of the device in use;

FIG. 3b shows a side view of the device in use;

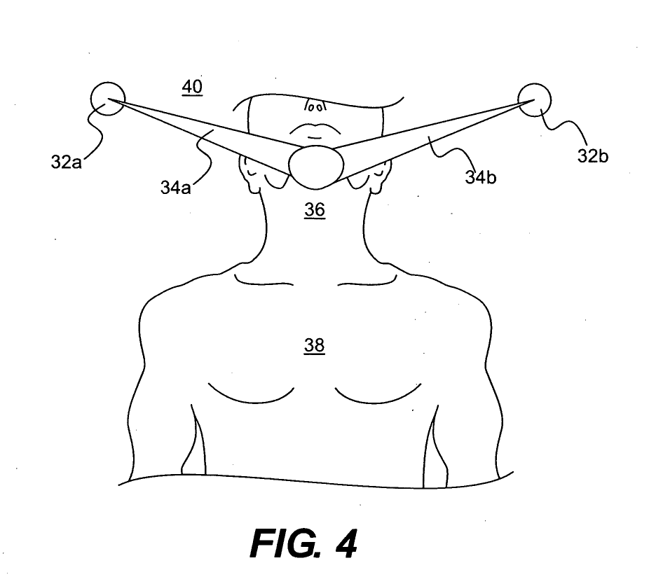

FIG. 4 shows an alternative embodiment in which the base is placed behind the head; and

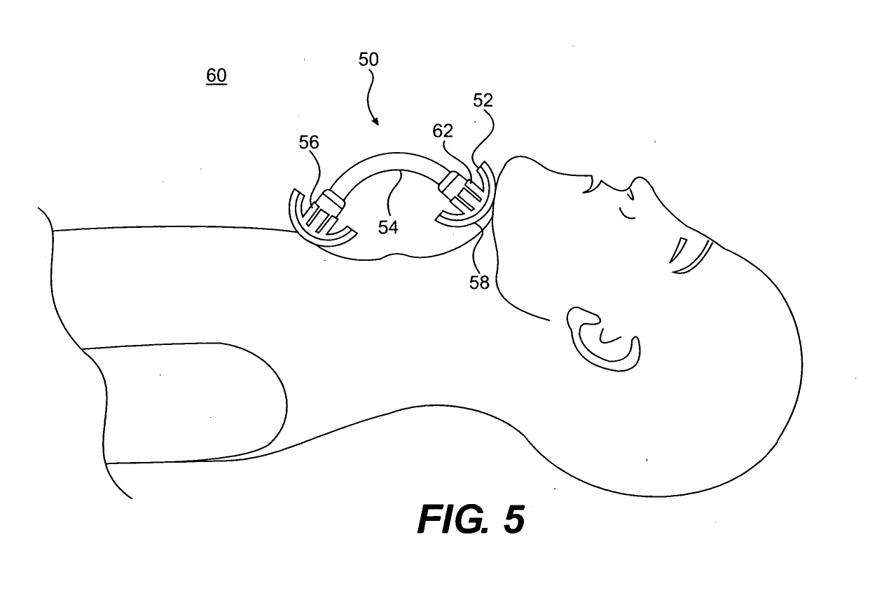

FIG. 5 is a perspective view of an embodiment of the invention with convex shaped chin support and base portions shown at the anticipated use angle.

### DETAILED DESCRIPTION OF THE INVENTION

Referring to FIGS. 1a-3b, wherein like numerals represent like parts throughout the several views, there is generally disclosed at 10 an embodiment of a hands free chin lift and airway support device. The hands free chin lift and airway support device herein referred to as device consists of three main components: the chinrest or chin support portion, the shaft or shaft support portion, and the base or base portion. In the embodiment of FIGS. 1a-3b, the components are made of a flexible thermoplastic elastomer such as polyethyleneterephthalate, available from Atofina Chemical, Inc. of Philadelphia, Pa. However, the components may consist of any other material that can be repeatedly bent and that can hold its bent shape without fracturing, such as polyethylene, polypropylene, vinyl, nylon, rubber, leather, various impregnated or laminated fibrous materials, various plasticized materials, cardboard, paper, etc.

In FIGS. 1a-3b, on one end of the device, referred to as proximal end 24, is the chin support portion 12. The chin rest is connected to a shaft support portion 14 at a first end which is connected at a second end to a base portion 16. The chin rest 12 comprises a material that will provide the user with the flexibility to mold the chin rest to fit the shape of the mandible of a patient. When in place the chin rest will hold shape throughout the medical procedure such that the device will not slip from the chin. In the embodiment of FIGS. 1a-3b, the chin rest is a ellipsoidal in shape. The shape of the chin rest is best described that of a pitted and halved avocado. This shape exerts pressure to hold the chin, coupled with an adhesive or friction surface to prevent the head from slipping out of the desired position. This unique shape allows support on both sides of a patient's mandible while encompassing the patient's chin to prevent the head from sliding out of a desired position 40. The chin rest is connected to the shaft 14 of the device by virtue of extrusion of liquid plastic.

At the point of contiguity of the chin rest and the shaft 18, the chin rest will be able to slightly bend up and down as shown in FIG. 1b. In the embodiment of FIGS. 1a-3b, the shaft 14 is a cylindrical solid that is malleable in order to accommodate a patient anatomy. The shaft is flared outward at both ends 20 to aid in structural integrity of the device. After the shaft is bent, the device will hold configuration throughout the procedure.

At one end of the device, referred to as distal end 22, the shaft will transfer the weight of a patient's head to the base 16. The shaft of the device is contiguous with the base and attached by virtue of extrusion of liquid plastic. The base 16 of the device is cylindrical in shape. The base 16 will hold the device in place on a patient's manubrium 38 such that the device will not move during use. This securement is achieved through the friction caused by the weight transferred from the head of the patient and/or through an adhesive, such as Uro-Bond® III Brush-On Adhesive available from Urocare® of Pomona, Calif. added to the bottom of the base 26.

While various sizes of the various components may be utilized, the device as shown has an overall length of 280 millimeters. The shaft of the device is 22.8 millimeters in length, and 51 millimeters in diameter. The base of the device is typically 127 millimeters in diameter and 12.7 millimeters high. The chin rest is half ellipsoid configuration with an inner 20 and outer 22 ellipses or concave. The inner long axis is 127 millimeters, and the inner short axis is 63.5 millimeters. The outer long axis is 133.4 millimeters, and the outer short axis is 70 millimeters. The sizes and figures stated herein are shown for an adult device. With different dimensions the same configuration of device can be used for infant, child or adolescent and are within the scope of the present invention.

FIGS. 3a-3b shows the device in use. In use, the distal end 22 of the device is placed approximately at the level of a patient's manubrium 38. The patient's head is positioned in the sniffing position with the mandible lifted upward such that good airway patency is confirmed by both visually observing the chest rise and fall and by feeling the patient's breath on the user's hand. The sniffing position is defined as the position of the head from neutral position rotated 90° facing front and fully abducted 40. The proximal end of the device 24 is then placed under the patient's chin. The device is then stabilized by adjusting the malleable shaft 14 and the chin rest 12 on the proximal end of the mandible. The airway patency is reevaluated. If a partial airway obstruction still exists, an oropharyngeal and/or nasopharyngeal is placed in the patient's oropharynx, and the device is repositioned as necessary. Confirmation of airway patency is noted, and the user will then continue to maintain vigilant monitoring of the patient airway throughout the procedure.

The FIG. 4 embodiment of the present invention includes two bases 32a and 32b. These bases may be poles, rails, hooks, etc. These bases will be positioned on both sides of and behind the patient's head. Two shafts 34a and 34b comprising of rope, tape, plastic, rubber, etc. will be attached to each base. At the midpoint of the shaft, the chinrest 36 will be placed on the patient chin. Tension will be used to hold the patient's head in the desired position.

The operation of the FIG. 4 embodiment includes that the bases 32a and 32b are positioned and secured behind the patient's head. The patient's head is placed in the sniffing position, and the chin rest 42 is placed on the mandible. The shafts 40 of the device are pulled taught to maintain the position of the head as needed.

Referring to FIG. 5, there is generally disclosed an embodiment of at 50 a hands free chin lift and airway support device. The hands free chin lift and airway support device 50 herein referred to as device consists of three main components: the chinrest 52 or chin support portion, the shaft 54 or shaft support portion, and the base 56 or base portion. In the embodiment of FIG. 5, the components are made of a flexible thermoplastic elastomer such as polyethyleneterephthalate, available from Atofina Chemical, Inc. of Philadelphia, Pa. However, the components may consist of any other material that can be repeatedly bent and that can hold its bent shape without fracturing, such as polyethylene, polypropylene, vinyl, nylon, rubber, leather, various impregnated or laminated fibrous materials, various plasticized materials, cardboard, paper, etc.

In FIG. 5, the chin rest 52 is connected to a shaft support portion 54 which is connected to a base portion 56. The chin support portion 52 has an upper convex portion 58 configured to be positioned under the patient's chin or sub-mental to exert upward pressure to thrust the chin forward so the patient's head position remains constant during movements that may occur during a medical procedure. The chin support portion 52 comprises a material that will provide stability of position. The chin support portion 52 provides a cushion coupled with friction or adhesion. When in place the chin support portion 52 rest will not slip from the chin. This unique shape prevents the head from sliding out of a desired position 60. The lower portion 62 of the chin support portion 52 is removably coupled to a first end of the shaft 54 of the device. The chin support portion 52 can be removed from the shaft support portion and disposed of and a new chin support portion can be removably coupled to the shaft.

In the FIG. 5 embodiment of the present invention, the shaft 54 will be a cylindrical solid that is malleable in order for the device 50 to accommodate a patient anatomy. After the shaft 50 is bent, the device will hold its configuration throughout the procedure. The shaft will transfer the weight of a patient's head to the base 56. The shaft of the device is configured to removably couple to the base. The base 56 can be removed from the shaft support portion and disposed of and a new base 56 can be removably coupled to the shaft.

In the FIG. 5 embodiment of the present invention, the base 56 of the device includes a lower convex portion that is configured to position on, and engage, the patient's chest and an upper portion configured to removably couple to a second end of the shaft support portion. The chin support portion 56 provides a cushion coupled with friction/adhesion. The base portion in the preferred embodiment will be anchored to the patient's chest both by adhesive and/or by the friction caused by the transfer of force from the patient's head to the chin support portion such that the device will not move during use.

In use, the base support portion 56 of the device is placed approximately at the level of a patient's manubrium 64. The patient's head is positioned in the sniffing position with the mandible lifted upward such that good airway patency is confirmed by both visually observing the chest rise and fall and by feeling the patient's breath on the user's hand. The sniffing position is defined as the position of the head from neutral position rotated 90° facing front and fully abducted 60. The convex upper portion of the chin support portion of the device 52 is then placed under and engages the patient's chin or sub-mental. The device is then stabilized by adjusting the malleable shaft 54 and the chin support portion 52. The airway patency is reevaluated. If a partial airway obstruction still exists, an oropharyngeal and/or nasopharyngeal is placed in the patient's oropharynx, and the device is repositioned as necessary. Confirmation of airway patency is noted, and the user will then continue to maintain vigilant monitoring of the patient airway throughout the procedure.

The present invention will prevent the need for an anesthetist to maintain continuous physical contact with the patient's mandible at all times. The anesthetist will be able to move freely as necessary as a result of not having to maintain continuous physical contact with the patient. The anesthetist will have the unimpeded ability to perform his or her other tasks, such as delivering medications and charting vital signs, during surgery as a result of not being forced to maintain continuous physical contact with the patient's mandible at all time. This results in a more efficient and less laborious performance of additional responsibilities. In addition, factors, such as operating room table placement and patient positioning, will have no effect on the anesthetist's ability to maintain constant pressure on the chin during long procedures. The anesthetist will not become unnecessarily fatigued and/or physically taxed as a result of laboriously maintaining constant pressure upon the patient's chin, but instead will be free to move to suit the needs of his/her body. Accordingly, the anesthesia provider will not have to resort to inducing general anesthesia to alleviate these difficulties. Thus, the negative effects of general anesthesia, such as potential for stroke, increased nausea, and injury to teeth may be avoided.

All patients undergoing TIVA anesthesia will have ability to have sterile, constant chin support. The anesthetist will have the ability to provide a clean disposable method for hands-free support of the mandible allowing proper alignment of anatomical structures of the airway, resulting in optimal air exchange, thereby preventing and/or resolving obstruction. The device will fit a variety of patient sizes, which will allow the anesthesia provider to use the device whenever necessary. The device or portions of the device will be disposable allowing the anesthesia provider to maintain clean sterile conditions for each patient.

Although the description above contains many specifications, these should not be construed as limiting the scope of the invention but as merely providing illustrations of some of the presently preferred embodiments of this invention. For example, the chin rest may be made of or contain a variety of different materials, such as gel padding, foam, tape, plastic, or any other material that provides comfort and mandible stability. The central malleable shaft may come in a variety of lengths and widths so as to fit an assortment of patient sizes; it may range from a piece of hollow or solid tubing to any predetermined shape made of any malleable material which can provide rigidity and which will maintain structural integrity. Additionally, the base shape, diameter, length, or adhesive properties may be increased or decreased as necessary to anchor the base to the patient's chest. The method of device adhesion should not be limited to adhesive, but may be tape, weight, gum, putty, pressure, friction, or any method that will provide comfort and ease of use while maintaining adhesive properties. Further, the components may comprise any other material that can be repeatedly bent and hold its bent shape without fracturing, such as but not limited to polyethylene, polypropylene, vinyl, nylon, rubber, leather, various impregnated or laminated fibrous materials, various plasticized materials, cardboard, and paper. The method of connecting the chin rest, shaft, and base is not limited to liquid plastic extrusion, but may be glue or other adhesive, solder, pin and rod assembly, welding, solvent-bonding, snap-fitting, threaded assembly, or any other method whereby the chin rest and base may be permanently or temporarily attached to the shaft.

Thus, the scope of the invention should be determined by the appended claims and their legal equivalents, rather than by the examples given.

What I claim is:

1. A system for assisting in the maintenance of patency to a patient's airway, comprising:
a malleable shaft support portion comprising a flexible material that will maintain its shape when bent;
a chinrest support portion comprising a material that includes an upper convex portion configured to position under the patient's chin, and a lower portion removably coupled to a first end of the shaft support portion; and
a base portion comprising a malleable material including an upper portion removably coupled to a second end of the shaft support portion and a lower portion configured for stable positioning on a patient's manubrium.

2. The system according to claim 1, wherein said base portion is configured to uncouple from the shaft portion.

3. The system according to claim 1, wherein said chin support portion is configured to uncouple from the shaft portion.

4. The system according to claim 1, wherein the upper convex portion of the chinrest support portion is configured with an adhesive having adherence to the skin at least sufficient to prevent the base portion from sliding on the patient's chest.

5. The system according to claim 1 wherein the shaft support portion, the chinrest support portion and base portion comprise a flexible thermoplastic elastomer planar.

6. The system according to claim 5, wherein the flexible thermoplastic elastomer is polyethyleneterephthalate.

7. The system according to claim 6, wherein the shape of the shaft support portion is one of: a cylinder, a circle, a rectangle, and a square.

8. The system according to claim 6, wherein the shape of the lower portion of the base portion is one of: a cylinder, a circle, a rectangle, a square and convex.

9. The system according to claim 1, wherein the base support portion is wider than the shaft support portion.

10. The system according to claim 1, wherein the shaft support portion, the chinrest support portion and base portion are made of one of: polyethylene, polypropylene, vinyl, nylon, rubber, leather, an impregnated material, a laminated fibrous material, a plasticized materials, cardboard and paper.

11. The system according to claim 1, further comprising a points of contiguity where the lower portion of the chin support portion and a first end of the shaft support portion are coupled and where the upper portion of the base and a second end of the shaft support portion are coupled.

12. The system according to claim 11, wherein the chin support portion and shaft support portion are adjustable at the point of contiguity.

13. The system according to claim 1, wherein the shaft support portion is bendable.

14. A method of assisting in the maintenance of patency to a patient's airway, comprising:
providing a malleable shaft support portion comprising a flexible material that will maintain its shape when bent;
providing a chinrest support portion comprising a material that includes an upper convex portion configured to position under the patient's chin, and a lower portion removably coupled to a first end of the shaft support portion; and
providing a base portion comprising a malleable material including an upper portion coupled to a second end of the shaft support portion and a lower portion configured for stable positioning on a patient's manubrium;
placing the base portion at the level of the patient's manubrium;
positioning the patient's head in the sniffing position; and
inserting the patient's chin in the chinrest support portion.

15. The method of claim 14, further comprising adjusting the shaft support portion and the chin rest support portion to maintain patency to the patient.

16. The method of claim 14, wherein the base portion includes a lower convex portion configured to position on, and engage the patient's manubrium.
